(19) United States
(12) Patent Application Publication
(10) Pub. No.: US 2017/0319822 A1
(43) Pub. Date: Nov. 9, 2017

(54) Intravenous Catheter Assembly

(71) Applicant: JIANGXI FENGLIN MEDICAL TECHNOLOGY CO.,LTD, Fengcheng City (CN)

(72) Inventor: Teoh Hwa ANG, Singapore (SG)

(21) Appl. No.: 15/522,278

(22) PCT Filed: Oct. 27, 2015
(86) PCT No.: PCT/SG2015/050409

§ 371 Date: Apr. 26, 2017

(30) Foreign Application Priority Data
Oct. 27, 2014 (SG) 10201406976S

(51) Int. Cl. A61M 25/00; A61M 39/06; A61M 25/06

(52) U.S. Cl. CPC A61M 25/0097; A61M 39/06; A61M 25/0606; A61M 25/0618; A61M 2039/062; A61M 2039/0666

(57) ABSTRACT

An intravenous catheter assembly comprises a catheter hub having an elongate axis and a lumen; a side port extending from the catheter hub, the side port being in fluid communication with the lumen; and a one-way port valve positioned in the side port, the one-way port valve having a valve portion formed from a resilient material. The valve portion can comprise a cap and a hinge portion about which the cap is rotatable. The cap can be disposed over an aperture formed in an inclined end face of the valve portion.

### FIELD OF THE INVENTION

The present invention relates to improvements in intravenous catheter assemblies.

### BACKGROUND OF THE INVENTION

Intravenous catheters (which may be central venous catheters or peripheral venous catheters, for example) are commonly used in situations when a patient requires frequent or continuous injections of medications or fluids. A cannula is inserted into a vein of the patient to provide a path for fluids to be injected. Typically, the cannula extends from a catheter hub which can be affixed to the patient's arm using adhesive. One type of intravenous (IV) catheter has a flexible cannula with a needle extending through it. The tip of the needle is used to make an incision into the patient's arm at the location of a vein, such that the cannula can be inserted into the vein. The needle acts as a guidewire for cannulation and is withdrawn once the cannula has been inserted. One problem which can occur during withdrawal is that due to high frictional forces between the needle and the rest of the catheter assembly, the entire assembly can be inadvertently pulled away from the vein. For example, in some catheter assemblies a resilient plug is provided in the catheter hub to prevent backflow. The resilient plug is formed from a springy material which encloses the needle. When the needle is withdrawn, the springy material closes the hole left by the withdrawal of the needle. Due to the strong compressive force required to keep a liquid-tight seal around the needle, there is a risk that withdrawing the needle can withdraw the whole assembly. One way to address this problem is to provide a lubricating gel between the needle and the plug, but this complicates the assembly process and does not always work.

This known type of IV catheter may have a transverse port branching off from the catheter hub, in order to allow injection of a medicament through the port, for example. In order to prevent backflow of blood and other fluids into the port a one-way valve may be deployed in the port. Typically a duckbill valve is provided in-line with the lumen of the catheter hub. It has been found that duckbill valves may not permit sufficient flow rate in some circumstances. In addition, because the catheter is stored in an

undeployed state with the needle protruding through the duckbill valve and placing it in an open (deformed) state, material fatigue may result in the two halves of the valve not being able to form an adequate seal when the needle is removed and the valve returns towards its closed (undeformed) state.

Typically, IV catheters include wings extending from the catheter hub, to enable gripping during cannulation, and also to provide a contact surface against the patient's hand. Because the wings are rigid to provide the gripping function, if the catheter is in place for an extended period, it can cause discomfort to the patient. To ease this discomfort, nurses may place plaster or other cushioning materials under the wings. However, this reduces the contact between the catheter and the patient's skin, thereby diminishing the ability of the catheter to stay secured to the patient's arm.

There remains a need for an IV catheter which can alleviate one or more of the above disadvantages, or at least provide a useful alternative.

### SUMMARY OF THE INVENTION

In some embodiments, the present invention relates to an intravenous catheter assembly, comprising:

a catheter hub having an elongate axis and a lumen;

a side port extending from the catheter hub, the side port being in fluid communication with the lumen; and

a one-way port valve positioned in the side port, the one-way port valve having a valve portion formed from a resilient material.

In certain embodiments, the valve portion comprises a cap and a hinge portion about which the cap is rotatable. The cap may be disposed over an aperture formed in an inclined end face of the valve portion.

In certain embodiments the catheter hub has a cannula extending therefrom, and the intravenous catheter assembly further comprises:

a needle housing removably attached to the catheter hub;

a needle disposed substantially along the elongate axis, the needle having a proximal end housed in the needle housing and a distal end sheathed by the cannula; and

a septum disposed within the lumen;

wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal.

The first and second resilient parts may each be in the form of a half-dome.

In certain embodiments the assembly further comprises a safety clip coupling the needle housing and the catheter hub, the safety clip having a housing having at least one sidewall, and a clip portion located within the housing, the clip portion having a catheter hub engagement portion; wherein, in an undeployed configuration of the catheter assembly, the clip portion is deflected by the needle such that the catheter hub engagement portion projects through the housing to engage the catheter hub; and in a deployed configuration when the needle has been withdrawn, the catheter hub engagement portion blocks a tip of the needle, and the tip of the needle is contained within an enclosure defined by the housing and the catheter hub engagement portion. The assembly may further comprise a deformable base member having a surface with a channel contoured to receive the catheter hub.

Some embodiments of the invention relate to an intravenous catheter assembly, comprising:

a catheter hub having an elongate axis and a lumen, the catheter hub having a cannula extending therefrom;

a needle housing removably attached to the catheter hub;

a needle disposed substantially along the elongate axis, the needle having a proximal end housed in the needle housing and a distal end sheathed by the cannula; and

a septum disposed within the lumen;

wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal.

The first and second resilient parts may each be in the form of a half-dome.

The assembly may further comprise a side port extending from the catheter hub, the side port being in fluid communication with the lumen; and a one-way port valve positioned in the side port, the one-way port valve having a valve portion formed from a resilient material. The valve portion may comprise a cap and a hinge portion about which the cap is rotatable. The cap may be disposed over an aperture formed in an inclined end face of the valve portion.

In certain embodiments the assembly further comprises a safety clip coupling the needle housing and the catheter hub, the safety clip having a housing having at least one sidewall, and a clip portion located within the housing, the clip portion having a catheter hub engagement portion; wherein, in an undeployed configuration of the catheter assembly, the clip portion is deflected by the needle such that the catheter hub engagement portion projects through the housing to engage the catheter hub; and in a deployed configuration when the needle has been withdrawn, the catheter hub engagement portion blocks a tip of the needle, and the tip of the needle is contained within an enclosure defined by the housing and the catheter hub engagement portion. The assembly may further comprise a deformable base member having a surface with a channel contoured to receive the catheter hub.

Some embodiments relate to an intravenous catheter assembly, comprising:

a catheter hub having an elongate axis and a lumen, the catheter hub having a cannula extending therefrom;

a needle housing removably attached to the catheter hub;

a needle disposed substantially along the elongate axis, the needle having a proximal end housed in the needle housing and a distal end sheathed by the cannula; and

a safety clip coupling the needle housing and the catheter hub, the safety clip having a housing having at least one sidewall, and a clip portion located within the housing, the clip portion having a catheter hub engagement portion;

wherein, in an undeployed configuration of the catheter assembly, the clip portion is deflected by the needle such that the catheter hub engagement portion projects through the housing to engage the catheter hub; and in a deployed configuration when the needle has been withdrawn, the catheter hub engagement portion blocks a tip of the needle, and the tip of the needle is contained within an enclosure defined by the housing and the catheter hub engagement portion.

The assembly may further comprise a septum disposed within the lumen; wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal. The first and second resilient parts may each be in the form of a half-dome.

The assembly may further comprise a side port extending from the catheter hub, the side port being in fluid communication with the lumen; and a one-way port valve positioned in the side port, the one-way port valve having a valve portion formed from a resilient material. The valve portion may comprise a cap and a hinge portion about which the cap is rotatable. The cap may be disposed over an aperture formed in an inclined end face of the valve portion.

The assembly may further comprise a deformable base member having a surface with a channel contoured to receive the catheter hub.

Some embodiments relate to an intravenous catheter assembly, comprising:

a catheter hub having an elongate axis and a lumen, the catheter hub having a cannula extending therefrom; and

a deformable base member having a surface with a channel contoured to receive the catheter hub.

The assembly may further comprise a side port extending from the catheter hub, the side port being in fluid communication with the lumen; and a one-way port valve positioned in the side port, the one-way port valve having a valve portion formed from a resilient material. The valve portion may comprise a cap and a hinge portion about which the cap is rotatable. The cap may be disposed over an aperture formed in an inclined end face of the valve portion.

In certain embodiments the catheter hub has a cannula extending therefrom, and the intravenous catheter assembly further comprises:

a needle housing removably attached to the catheter hub;

a needle disposed substantially along the elongate axis, the needle having a proximal end housed in the needle housing and a distal end sheathed by the cannula; and

a septum disposed within the lumen;

wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal.

In certain embodiments, the first and second resilient parts are each in the form of a half-dome.

Certain embodiments may comprise a safety clip coupling the needle housing and the catheter hub, the safety clip having a housing having at least one sidewall, and a clip portion located within the housing, the clip portion having a catheter hub engagement portion; wherein, in an undeployed configuration of the catheter assembly, the clip portion is deflected by the needle such that the catheter hub engagement portion projects through the housing to engage the catheter hub; and in a deployed configuration when the needle has been withdrawn, the catheter hub engagement portion blocks a tip of the needle, and the tip of the needle is contained within an enclosure defined by the housing and the catheter hub engagement portion.

Some embodiments relate to a one-way valve for a side port of an intravenous catheter assembly, the one-way port valve having a valve portion formed from a resilient

material, the valve portion comprising a cap and a hinge portion about which the cap is rotatable. The cap may be disposed over an aperture formed in an inclined end face of the valve portion.

Some embodiments relate to a safety clip for coupling a needle housing and a catheter hub of an intravenous catheter assembly, the safety clip comprising:

a housing having at least one sidewall, and a clip portion located within the housing, the clip portion having a catheter hub engagement portion;

wherein the clip portion is deflectable to an engagement position such that the catheter hub engagement portion projects through the housing for engaging the catheter hub; and wherein the clip portion is movable to a deployed configuration in which the catheter hub engagement portion is located within the housing to define, together with the at least one sidewall, an enclosure for a needle tip.

### BRIEF DESCRIPTION OF THE INVENTION

Embodiments of the invention will now be described, by way of non-limiting example only, with reference to the accompanying drawings in which:

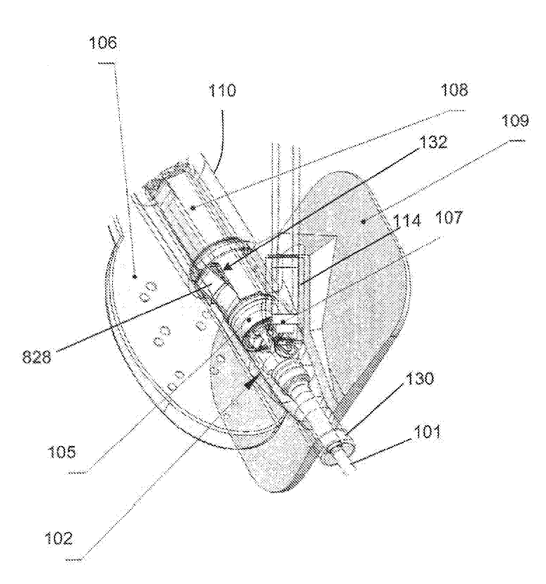

FIG. 1 is a perspective view of an intravenous (IV) catheter assembly according to some embodiments of the invention;

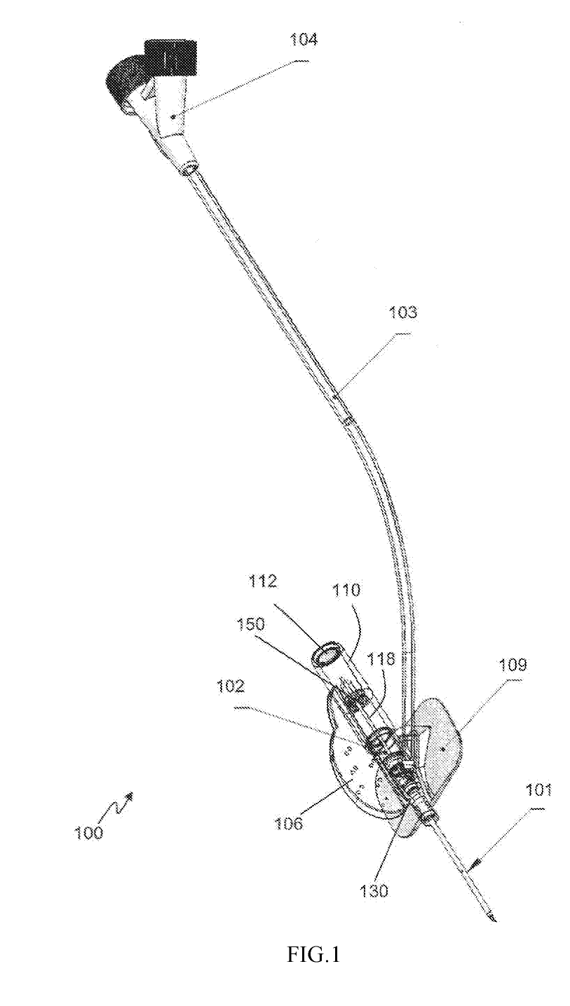

FIG. 2 is a close-up view of part of the IV catheter assembly of FIG. 1;

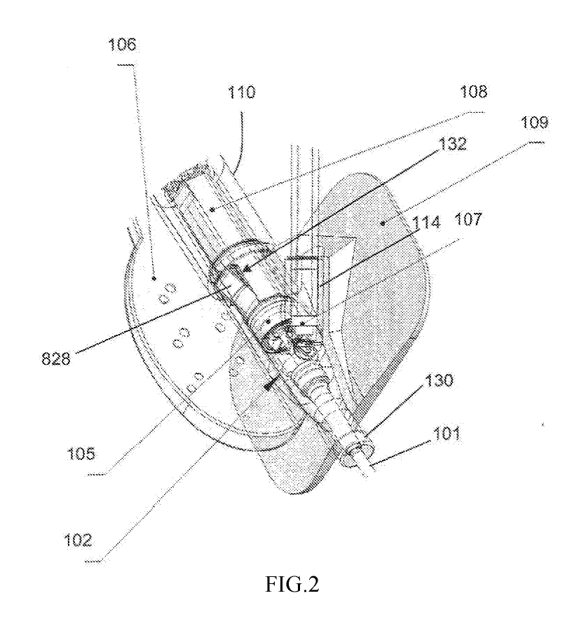

FIG. 3 is a side view of the IV catheter assembly;

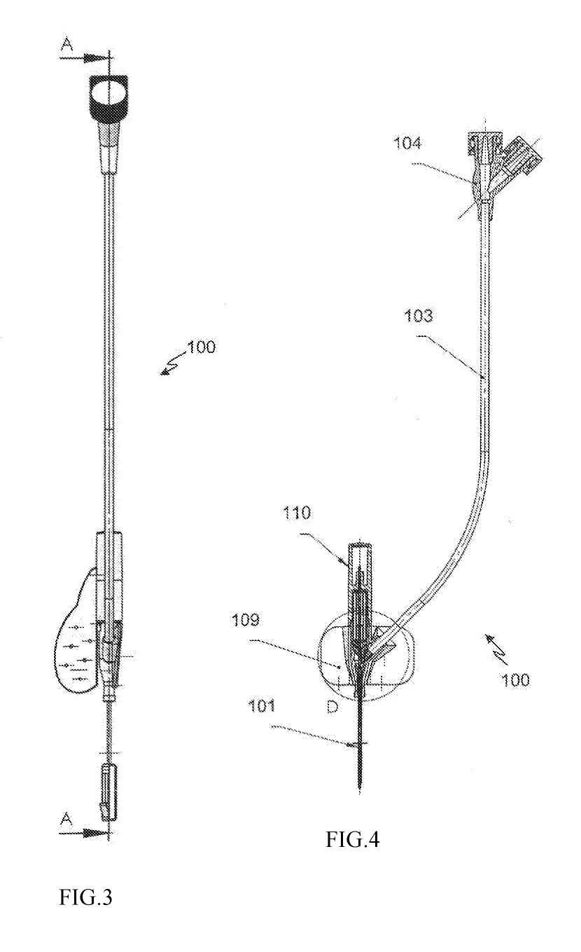

FIG. 4 is a cross-section through the line A-A of FIG. 3;

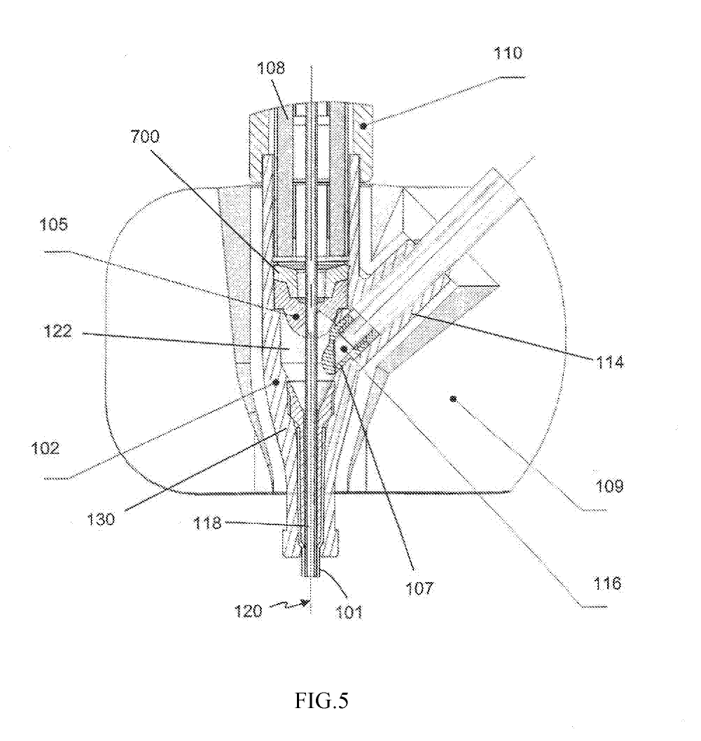

FIG. 5 is a detail at D of FIG. 4;

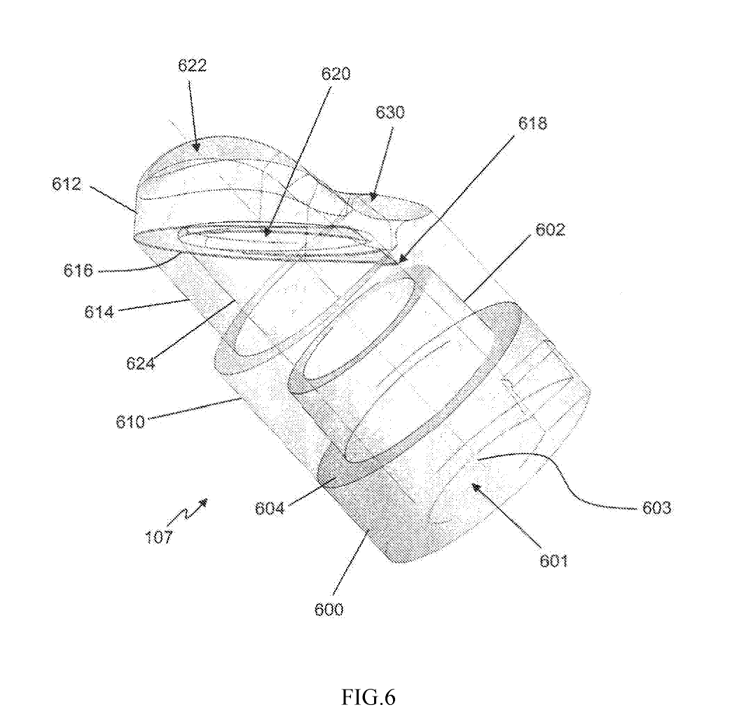

FIG. 6 is a perspective view of a one-way port valve of the IV catheter assembly;

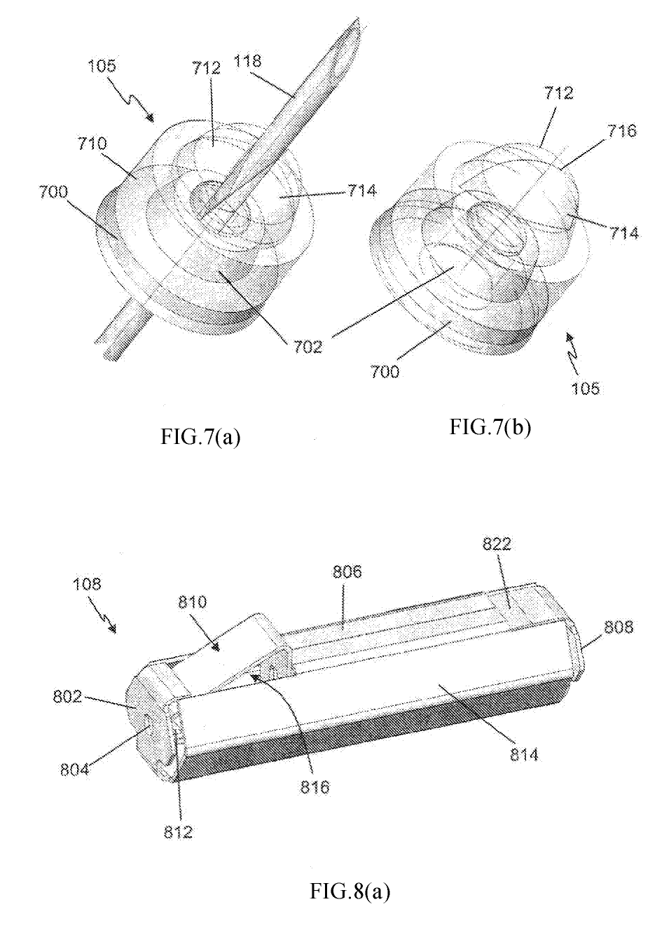

FIG. 7(a) is a perspective view of a dome septum of the IV catheter assembly in an open configuration with a needle projecting therethrough;

FIG. 7(b) shows the dome septum in a closed configuration;

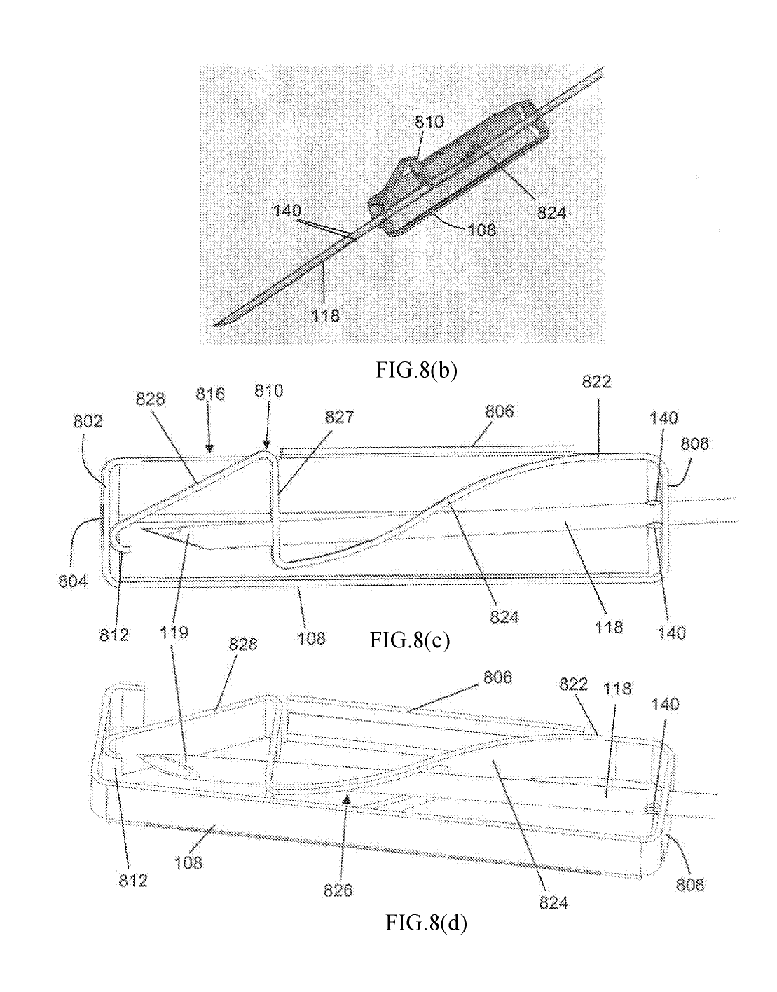

FIG. 8(a) is a perspective view of a safety clip of the IV catheter assembly;

FIG. 8(b) is a perspective cutaway view of the safety clip in place on a needle;

FIG. 8(c) is a side cutaway view of the safety clip after the needle has been withdrawn;

FIG. 8(d) is a bottom perspective cutaway view of the safety clip with needle withdrawn;

FIG. 8(e) is a perspective view of a needle holder and needle of the catheter assembly after withdrawal of the needle, with the safety clip in place; and

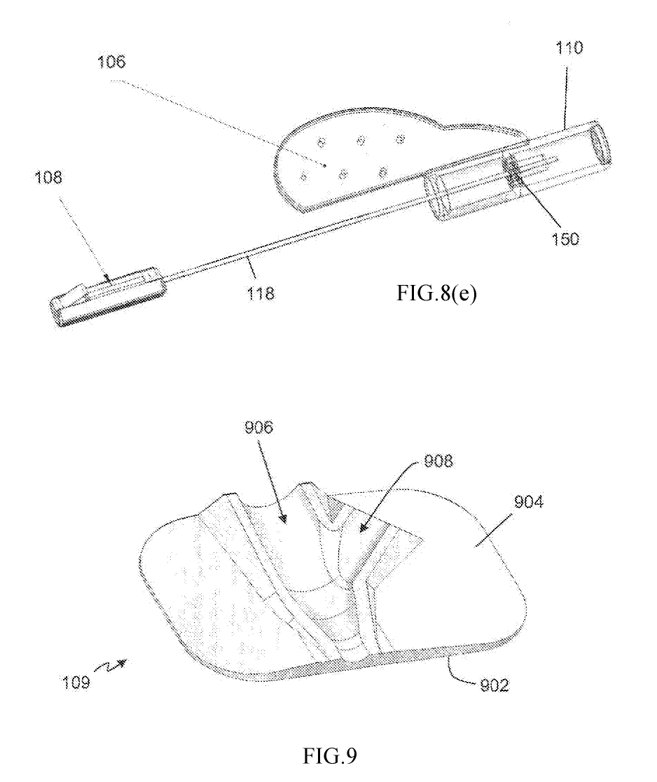

FIG. 9 is a top perspective view of a base member of the IV catheter assembly.

### DETAILED DESCRIPTION OF INVENTION

Referring to FIGS. 1 to 5, there is shown an IV catheter assembly 100 comprising a cannula 101, which projects from a catheter hub 102 along a longitudinal axis 120 (as shown in FIG. 5) of the hub 102. In the undeployed configuration shown in FIGS. 1 to 5, a needle 118 projects through the cannula 101. The cannula 101 is preferably a soft tube which sheaths and is guided by the rigid needle 118 during a cannulation procedure. The needle 118 may have a bevelled tip in order to make an incision at the site of a patient's vein so that the cannula 101 can be inserted into the vein, guided by needle 118. The needle 118 is then withdrawn in order to leave cannula 101 in the vein.

The catheter hub 102 comprises a main body portion 130 having a lumen 122. A port 114 extends laterally from the main body portion 130 and has an end portion 116 which is in fluid communication with the lumen 122 via a one-way port valve 107. After the needle 118 has been withdrawn, the port 114 functions to allow injection of a fluid, such as a fluid containing a medicament, into the lumen 122 and thus into the cannula 101. The fluid may be injected along an extension tube 103 which is coupled to the port 114, and the extension tube 103 may be coupled at the other end to a Y-connector 104 to allow mixing of two fluids (e.g., an active ingredient and a diluent) prior to injection into port 114. Other types of connector having one or more junctions for mixing two or more fluids may be used.

The catheter hub 102 may be arranged on a base member 109 which is contoured to receive the main body portion 30 and the port 114. For example, as shown in FIG. 9, the base member may have a lower surface 902 which is relatively flat, and an upper surface 904 on which are provided a first contoured channel 906 to receive the main body portion 130, and a second contoured channel 908, branching off from the first contoured channel 906, to receive the port 114.

In some embodiments the base member 109 may be adhered (either permanently or temporarily) to the catheter hub 102. In other embodiments it may be provided as a separate member which is placed between the catheter hub 102 and the patient's skin during installation of the catheter 100.

The base member 109 is formed of a deformable material, such as a medical grade elastomeric material, so that it can conform to the surface contours of the patient's skin at the point of installation. This increases the contact area between the base member 109 and the patient's skin, compared to the rigid assemblies of known IV catheter hubs, helping to ensure that the catheter stays in place. Further, if a suitably soft and/or springy material is chosen for the base member 109, the catheter may stay in place without causing significant discomfort to the patient. The material of base member 109 may also be chosen to be relatively sticky such that friction between the base member 109 and the patient's skin is increased relative to either the rigid assemblies of the prior art or other makeshift solutions such as cloth bandages or padding. The base member 109 may be formed from a silicone-based material, such as a USP class Vl-compliant elastomer, for example.

The catheter hub 102 is attached, in the undeployed configuration, to a detachable needle housing 110 which can be gripped by a handle 106 and pulled to detach the needle housing 110 from the catheter hub 102. A proximal end of the needle 118 is attached to the needle housing 110 at an attachment point 150, for example by bonding using epoxy or any other suitable adhesive.

The term “proximal”, as used herein, refers to parts or directions which, in use of the IV catheter assembly, would be closer to a person (e.g., a nurse) inserting the catheter than to the patient into whom the catheter was being inserted, while the term “distal” refers to parts or directions which would be further away from the person installing the catheter than the patient. For example, the tip of needle 118 or the cannula 101 of the catheter assembly 100 would be “distal” components while the needle housing 110 would be a “proximal” component. Likewise, the “proximal” end of the needle 118 would be the end which is closer to the person inserting the catheter than the needle tip.

The attachment of catheter hub 102 to needle housing 110 is by way of a safety clip 108 which has one end located partly within the housing 110 and another end located partly within the catheter hub 102. Preferably, as shown in FIG. 5, the safety clip 108 has an elongate body which, in the undeployed configuration, resides partly within the needle housing 110 and partly within the catheter hub 102, extending into the catheter hub 102 such that its front end 802 (FIG. 8(a)) abuts an end surface of a catheter hub valve 105. A catheter hub engagement portion 828 of the safety clip 108 protrudes through a window 132 in a surface of the catheter hub 102. The catheter hub engagement portion 828 prevents movement of the catheter hub 102 relative to the needle housing 110 until the safety clip 108 is released, as will later be described.

When the needle housing 110 is pulled away from the catheter hub 102, the tip of needle 118 is withdrawn through the catheter 101 and into safety clip 108. The detachable housing 110, safety clip 108 and needle 118 (FIG. 8(e)) may then be disposed of.

In some embodiments, the IV catheter assembly 100 may comprise a septum 105 through which needle 118 passes. The septum 105 has first and second resilient parts which are separated by the needle 118 when the catheter assembly is in an undeployed configuration. Preferably, the first and second resilient parts are each in the shape of a half-dome, such that when the needle 118 is withdrawn, the first and second parts together form a dome which seals the septum. By using a dome septum it is possible to prevent backflow of fluids in the catheter hub lumen 122 whilst also minimising the contact forces between the needle 118 and septum 105, such that the possibility of withdrawing the catheter hub 102 when the needle 118 is withdrawn is greatly reduced. The dome septum 105 provides a point contact with the needle 118, as shown in FIG. 7(a), in which the needle is shown in a partially withdrawn state after cannulation. This has much lower friction than the circumferential contact provided by prior art valve structures.

In the particular construction shown in FIG. 7(a), the dome septum 105 comprises a rigid base member 700 on which a resilient valve member 710 is disposed. In particular, the rigid base member 700 may be coupled to the valve member 710 by a friction fit, with boss 702 fitting to a corresponding mating surface of the valve member 710. The valve member 710 may have an upper portion with a substantially hemispheric profile, and having two half-hemispheres 712 and 714 joined at seal 716. The valve member 710 is formed of a resilient material, such as a medical grade elastomeric material, such that when needle 118 is fully withdrawn, the half-hemispheres 712 and 714 spring back together and form a liquid-tight seal at seal 716. The dome-shaped structure formed by hemispheres 712 and 714 provides a greater range of movement than alternative structures, thus ensuring that there is greater spring back, and a tighter seal, when the needle 118 is withdrawn.

In some embodiments, a one-way valve 107 is provided in the port 114. The one-way port valve 107 is illustrated in more detail in FIG. 6. It may comprise a relatively rigid base portion 600 on which is mounted a resilient valve member 610. For example, the resilient valve member 610 may be seated on an annular ledge 604 defined about a boss 602 of the rigid base portion 600, and a friction fit may be formed between the resilient valve member 610 and the boss 602. The resilient valve member 610 is preferably formed from a medical grade elastomeric material.

The resilient valve member 610 has a head portion 614 which is sealed by a cap 612 at join 616. The cap 612 can be forced away from join 616 if sufficient fluid pressure is received via bore 624. This causes cap 612 to swing open about a hinge portion 618 such that fluid can flow through an angled aperture 620 at the opening of bore 624. Because the head portion 614 is resilient, once the fluid pressure is no longer being applied, cap 612 will close and there will be no opportunity for fluid in lumen 122 to flow back through port 114.

The valve 107 has a tubular channel, generally indicated at 601, which is defined by the inner surfaces of boss 602 and bore 624 of head portion 614. The tubular channel 601 has a longitudinal axis 603 which, when the valve 107 is installed in the port 114, is generally aligned with a corresponding longitudinal axis of the port 114 (and thus with tubing 103 inserted in the port 114, for example). The tubular channel 601 terminates at an aperture 620 having a plane which is inclined with respect to the normal to longitudinal axis 603.

Advantageously, by having an angled aperture 620, it is possible to increase the surface area at the aperture. This means that the force applied to the cap 612 at a given pressure will be increased, so that greater flow rates are possible at lower pressures. The angle is preferably 45°, but other choices of angle are of course possible as will be appreciated by those of ordinary skill in the art.

The cap portion 612 is preferably of relatively thick construction so as to be able to resist collapse due to fluid backpressure. A scalloped portion 622 may be provided in cap 612 to reduce the amount of material required, whilst still providing the requisite backpressure resistance. Preferably, a region 630 of reduced thickness is provided in the vicinity of hinge 618, in order to further reduce the fluid pressure required to be supplied through port 114 to open the cap 612 about hinge 618.

The catheter assembly 100 may advantageously comprise a safety clip 108, as previously alluded to and as shown in detail in FIG. 8(a) to FIG. 8(d). The safety clip has an elongate body defined by a distal end plate 802 having a first aperture 804 formed therein, sidewalls 814, and a proximal end plate 808 having a second aperture 809 formed therein. The first aperture 804is sized and shaped to receive the needle 1 18 and is wide enough to accommodate a pair of stop members 140 located on the needle barrel. On the other hand, the second aperture 809 is sized and shaped to accommodate the needle barrel, but is narrower than the combined width of the needle barrel diameter and the two stop members 140, such that the needle 118 is prevented from being withdrawn any further once the stop members 140 abut against proximal end plate 808.

The safety clip 108 also has a resilient clip portion 810 which extends from proximal end plate 808 to distal end plate 802. The resilient clip portion 810 has a catheter hub engagement portion 828 which, when the catheter assembly 100 is in an undeployed configuration, projects upwardly into the gap 816 between the top wall 806 and the distal end plate 802 of the safety clip 108. In the undeployed configuration, the gap 816 is generally aligned with the window 132 in the catheter hub such that the catheter hub engagement portion 828 also projects through the window 132 to secure the needle holder 110 to the catheter hub 102 as previously described. In particular, an engagement surface 827 of the catheter hub engagement portion 828 abuts against the catheter hub 102.

The clip portion 810 has a first portion 822 which descends from top wall 806 towards a lower wall of the safety clip 108 to define an intermediate spring portion 824 which has a channel 826 to receive the needle 118. The clip portion 810 terminates in catheter hub engagement portion 828 which protrudes above top wall 806 when the catheter 100 is yet to be deployed, as described above, and which has a finger 812 at a lower end thereof.

When the catheter assembly 100 is in the undeployed state, as shown in FIG. 8(b) (in which the catheter hub 102 and needle holder 110 are omitted for clarity), the needle 1 18 projects through second aperture 809, channel 826, under the finger 812 of catheter hub engagement portion 828, and through the first aperture 804. In this configuration, the end portion 828 is deflected upwardly by needle 118, due to the reaction force provided to the finger 812 by the needle 118 as it spans between the apertures 804 and 809. After cannuiation has been completed and the needle 118 is withdrawn, the finger 812 acts as a mechanical sensor, such that as needle tip 119 is withdrawn past finger 812, the needle 118 can no longer provide a reaction force, and the catheter hub engagement portion 828 moves downwardly such that the finger 812 is now positioned in front of the needle tip 119 to catch the tip 119 and prevent it from pushing out through aperture 804. In addition, stop members 140 abut against proximal end plate 808 to prevent further withdrawal of the needle 118. Accordingly, the needle tip 119 is retained within safety clip 108, as shown in FIG. 8(e). Advantageously, by providing a safety clip body with an enclosure for surrounding the needle tip 119, blood retained in the end of needle 118 (needle flash) is not made visible to the patient, and the enclosure minimises the risk of egress of blood droplets, which tend to be retained against the internal surfaces of the enclosure by surface tension.

The safety clip 108 is preferably formed by progressive stamping. For example, a plurality of templates may be cut from a metal sheet, and each template may then undergo a series of stamping operations to progressively define the sidewalls 814, clip portion 810, end plates 802 and 808, and top wall 806.

Whilst the foregoing description has described exemplary embodiments, it will be understood by those skilled in the technology concerned that many variations in details of design, construction and/or operation may be made without departing from the present invention.

What is claimed is:

1. An intravenous catheter assembly, comprising:
a catheter hub having an elongate axis and a lumen;
a side port extending from the catheter hub, the side port being in fluid communication with the lumen; and
a one-way port valve positioned in the side port, the one-way port valve having a valve portion formed from a resilient material.

2. The intravenous catheter assembly of claim 1, wherein the valve portion comprises a cap and a hinge portion about which the cap is rotatable.

3. The intravenous catheter assembly of claim 2, wherein the cap is disposed over an aperture formed in an inclined end face of the valve portion.

4. The intravenous catheter assembly of claim 1, wherein the catheter hub has a cannula extending therefrom, and wherein the intravenous catheter assembly further comprises:
a needle housing removably attached to the catheter hub;
a needle disposed substantially along the elongate axis, the needle having a proximal end housed in the needle housing and a distal end sheathed by the cannula; and
a septum disposed within the lumen;
wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal.

5. The intravenous catheter assembly of claim 4, wherein the first and second resilient parts are each in the form of a half-dome.

6. The intravenous catheter assembly of claim 4, further comprising:
a safety clip coupling the needle housing and the catheter hub, the safety clip having a housing having at least one sidewall and a clip portion located within the housing, the clip portion having a catheter hub engagement portion;
wherein in an undeployed configuration of the catheter assembly, the clip portion is deflected by the needle such that the catheter hub engagement portion projects through the housing to engage the catheter hub; and
in a deployed configuration when the needle has been withdrawn, the catheter hub engagement portion blocks a tip of the needle, and the tip of the needle is contained within an enclosure defined by the housing and the catheter hub engagement portion.

7. The intravenous catheter assembly of claiml, further comprising:
a deformable base member having a surface with a channel contoured to receive the catheter hub.

8. An intravenous catheter assembly, comprising:
a catheter hub having an elongate axis and a lumen, the catheter hub having a cannula extending therefrom;
a needle housing removably attached to the catheter hub;
a needle disposed substantially along the elongate axis, the needle having a proximal end housed in the needle housing and a distal end sheathed by the cannula; and
a septum disposed within the lumen;
wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal.

9. The intravenous catheter assembly of claim 8, wherein the first and second resilient parts are each in the form of a half-dome.

10. The intravenous catheter assembly of claim 8, further comprising:
a side port extending from the catheter hub, the side port being in fluid communication with the lumen; and
a one-way port valve positioned in the side port, the one-way port valve having a valve portion formed from a resilient material.

11. The intravenous catheter assembly of claim 10, wherein the valve portion comprises a cap and a hinge portion about which the cap is rotatable.

12. The intravenous catheter assembly of claim 11, wherein the cap isdisposed over an aperture formed in an inclined end face of the valve portion.

13. The intravenous catheter assembly of claim 8, further comprising:
a safety clip coupling the needle housing and the catheter hub, the safety clip having a housing having at least one sidewall, and a clip portion located within the housing, the clip portion having a catheter hub engagement portion;
wherein in an undeployed configuration of the catheter assembly, the clip portion is deflected by the needle such that the catheter hub engagement portion projects through the housing to engage the catheter hub; and
in a deployed configuration when the needle has been withdrawn, the catheter hub engagement portion blocks a tip of the needle, and the tip of the needle is contained within an enclosure defined by the housing and the catheter hub engagement portion.

14. The intravenous catheter assembly of claim 8, further comprising:
a deformable base member having a surface with a channel contoured to receive the catheter hub.

15. An intravenous catheter assembly, comprising:
a catheter hub having an elongate axis and a lumen, the catheter hub having a cannula extending therefrom;
a needle housing removably attached to the catheter hub;
a needle disposed substantially along the elongate axis, the needle having a proximal end housed in the needle housing and a distal end sheathed by the cannula; and
a safety clip coupling the needle housing and the catheter hub, the safety clip having a housing having at least one sidewall, and a clip portion located within the housing, the clip portion having a catheter hub engagement portion;
wherein, in an undeployed configuration of the catheter assembly, the clip portion is deflected by the needle such that the catheter hub engagement portion projects through the housing to engage the catheter hub; and in a deployed configuration when the needle has been withdrawn, the catheter hub engagement portion blocks a tip ofthe needle, and the tip of the needle is contained within an enclosure defined by the housing and the catheter hub engagement portion.

16. The intravenous catheter assembly of claim 15, further comprising:
a septum disposed within the lumen; wherein in an undeployed configuration of the intravenous catheter assembly, the needle extends through the septum to separate first and second resilient parts of the septum; and
in a deployed configuration in which the needle housing has been detached and the needle has been withdrawn, the first and second resilient parts abut each other to form a liquid-tight seal.

17. The intravenous catheter assembly of claim 16, wherein the first and second resilient parts are each in the form of a half-dome.

18. The intravenous catheter assembly of claim15, further comprising:
a side port extending from the catheter hub, the side port being in fluid communication with the lumen; and
a one-way port valve positioned in the side port, the one-way port valve having a valve portion formed from a resilient material.

19. The intravenous catheter assembly of claim 18, wherein the valve portion comprises a cap and a hinge portion about which the cap is rotatable.

20. The intravenous catheter assembly of claim 19, wherein the cap is disposed over an aperture formed in an inclined end face of the valve portion.

21. The intravenous catheter assembly according to claim 15, further comprising a deformable base member having a surface with a channel contoured to receive the catheter hub.

22. An intravenous catheter assembly, comprising:
a catheter hub having an elongate axis and a lumen, the catheter hub having a cannula extending therefrom; and
a deformable base member having a surface with a channel contoured to receive the catheter hub.

23. The intravenous catheter assembly of claim 22, further comprising:
a side port extending from the catheter hub, the side port being in fluid communication with the lumen; and
a one-way port valve positioned in the side port, the one-way port valve having a valve portion formed from a resilient material.

24. The intravenous catheter assembly of claim 23, wherein the valve portion comprises a cap and a hinge portion about which the cap is rotatable.

25. The intravenous catheter assembly of claim 24, wherein the cap is disposed over an aperture formed in an inclined end face of the valve portion.

26. The intravenous catheter assembly of claim22, wherein the catheter hub has a cannula extending therefrom, and wherein the intravenous catheter assembly further comprises:
a needle housing removably attached to the catheter hub;
a needle disposed substantially along the elongate axis, the needle having a proximal end housed in the needle housing and a distal end sheathed by the cannula; and
a septum disposed within the lumen;
wherein in an undeployed configuration of the intravenous catheter assembly, the needle extends through the septum to separate first and second resilient parts of the septum, and in a deployed configuration in which the needle housing has been detached and the needle has been withdrawn, the first and second resilient parts abut each other to form a liquid-tight seal.

27. The intravenous catheter assembly of claim 26, wherein the first and second resilient parts are each in the form of a half-dome.

28. The intravenous catheter assembly of claim 22, further comprising:
a safety clip coupling the needle housing and the catheter hub, the safety clip having a housing having at least one sidewall, and a clip portion located within the housing, the clip portion having a catheter hub engagement portion;
wherein, in an undeployed configuration of the catheter assembly, the clip portion is deflected by the needle such that the catheter hub engagement portion projects through the housing to engage the catheter hub; and in a deployed configuration when the needle has been withdrawn, the catheter hub engagement portion blocks a tip of the needle, and the tip of the needle is contained within an enclosure defined by the housing and the catheter hub engagement portion.

29. A one-way valve for a side port of an intravenous catheter assembly, the one-way port valve having a valve portion formed from a resilient material, the valve portion comprising a cap and a hinge portion about which the cap is rotatable.

30. The one-way valve of claim 29, wherein the cap is disposed over an aperture formed in an inclined end face of the valve portion.

31. A safety clip for coupling a needle housing and a catheter hub of an intravenous catheter assembly, the safety clip comprising:
a housing having at least one sidewall, and a clip portion located within the housing, the clip portion having a catheter hub engagement portion;
wherein the clip portion is deflectable to an engagement position such that the catheter hub engagement portion projects through the housing for engaging the catheter hub; and wherein the clip portion is movable to a deployed configuration in which the catheter hub engagement portion is located within the housing to define, together with the at least one sidewall, an enclosure for a needle tip.
